(19) United States
(12) Patent Application Publication
(10) Pub. No.: US 2009/0032019 A1
(43) Pub. Date: Feb. 5, 2009

(54) ONE-PIECE, FOLDABLE MASK AND HOLDING CHAMBER FOR USE WITH AEROSOLIZED MEDICATIONS

(76) Inventors: Eric Matthew Green, Palo Alto, CA (US); Barry Nathan Wohl, Richmond (CA); Santiago Ocejo-Torres, Leon (MX)

Correspondence Address: MAYER BROWN LLP, P.O. BOX 2828, CHICAGO, IL, 60690, US

(21) Appl. No.: 12/182,700

(22) Filed: Jul. 30, 2008

Related U.S. Application Data
(60) Provisional application No. 60/962,504, filed on Jul. 30, 2007.

(51) Int. Cl. A61M 16/10

(52) U.S. Cl. 128/203.29

(57) ABSTRACT

A one-piece, foldable mask and holding chamber for use with aerosolized medications includes a single flat patterned sheet of formable material that can be bent and folded to form a holding chamber, an integrated respiration mask, a one-way mask inlet valve having an attached valve membrane, and a one-way exhalation outlet valve having an attached valve membrane. The apparatus is configured to form a substantial seal around the nose and mouth of a patient such that the patient can inhale an aerosolized medication that has been introduced into the holding chamber by drawing the medication from the holding chamber, through the inlet valve to the interior of the mask, and into the lungs of the patient. The apparatus is formed by folding a series of segments of the flat pattern along various crease lines to create the holding chamber and mask, and then securing the segments in place with a series of retention tabs and tab engagement slots.

### CROSS-REFERENCE TO RELATED APPLICATIONS

This application claims priority to U.S. Provisional Application Ser. No. 60/962,504 filed Jul. 30, 2007, the entire contents of which is hereby incorporated by reference herein.

### FIELD OF THE INVENTION

This disclosure relates to a foldable mask and holding chamber apparatus to aid individuals in the inhalation of aerosolized medications.

### BACKGROUND

Medication can be delivered to a patient's lungs to help treat various respiratory diseases such as asthma by putting it into aerosol particles, and then ensuring that the patient inhales these aerosolized medication particles. Numerous devices exist to create aerosolized medication particles, such as metered-dose inhalers (MDI's) and nebulizers. However, for these devices to work effectively, the production of aerosolized medication particles must be synchronized in time and space with the patient's inhalation of the aerosolized medication particles. This is a significant challenge, particularly for young children in respiratory distress, such as those suffering from an asthma attack. This challenge has been addressed by combining devices that generate medicated aerosols with two additional components: (1) a holding chamber, or “spacer” as it is known in the industry, which captures the aerosolized medication and dispenses it only when the patient inhales; and (2) a mask that connects to the spacer and channels the medication from the spacer's interior into the child's lungs.

This system is effective, but existing versions are too expensive, often costing as much as $50 or $60 USD, for much of the patient population to have each patient be able to afford to purchase and use his own mask and spacer system. This is especially difficult for those individuals in poorer, less economically developed countries where the patient often lives rural areas, far from referral hospitals or clinics. This is one major drawback of the existing systems available today. Additionally, the bulkiness of existing spacers, which are often made of hard plastic or metal tubes, and accompanying masks makes them inconvenient for patients to carry with them or for manufacturers to distribute. This is yet another significant drawback of existing systems. These drawbacks leave a significant unmet need for a compact and very inexpensive device that incorporates a holding chamber and mask to facilitate the delivery of aerosolized medication to the lungs of an individual when patients are suffering from an attack of an acute respiratory disease such as an asthma attack.

### SUMMARY

Described herein is a one-piece, foldable mask and holding chamber apparatus for use with aerosolized medications, as well as a method of assembling such an apparatus from a single, flat-patterned sheet of durable material.

The apparatus preferably includes a formable flat-patterned sheet of durable material that is formed to bound a substantially closed aerosolized medication holding chamber having a first end and a second end, and a respiration mask integrally connected to the second end of the holding chamber. An aerosolized medication insertion port is disposed in the first end of the holding chamber and is configured to receive a device for releasing aerosolized medications. A one-way mask inlet valve is disposed in a common dividing wall between the second end of the holding chamber and the respiration mask, for providing a passage for the aerosolized medications from the chamber into the mask. In addition, a one-way mask exhalation outlet valve is disposed in the mask for allowing exhalations from the patient's nose or mouth to escape to a surrounding environment, external to the apparatus.

Other embodiments, objects, features and advantages will be set forth in the detailed description of the embodiments that follows, and in part will be apparent from the description, or may be learned by practice, of the claimed invention. These objects and advantages will be realized and attained by the processes and compositions particularly pointed out in the written description and claims hereof. The foregoing Summary has been made with the understanding that it is to be considered as a brief and general synopsis of some of the embodiments disclosed herein, is provided solely for the benefit and convenience of the reader, and is not intended to limit in any manner the scope, or range of equivalents, to which the appended claims are lawfully entitled.

### BRIEF DESCRIPTION OF THE DRAWINGS

FIG. 1 is an isometric view of the preferred embodiment in use in its assembled, functional form, wherein a mask covers a patient's mouth and nose, and aerosolized medication is introduced through an aerosolized medication insertion port disposed in a first end-wall of a holding chamber.

FIG. 2A is a cross-sectional view of the preferred embodiment in use in its assembled, functional form, illustrating the introduction of aerosolized medication into the holding chamber of the assembled apparatus.

FIG. 2B is a cross-sectional view of the preferred embodiment in use in its assembled, functional form, illustrating a patient's inhalation of the aerosolized medication from the holding chamber, whereby a patient's inhalation causes the one-way mask exhalation outlet valve to close and the one-way mask inlet valve to open, allowing the medication to enter the mask and be inhaled into a patient's lungs through his mouth and nose.

FIG. 2C is a cross sectional view of the preferred embodiment in use in its assembled, functional form, illustrating the exhalation of the patient following the inhalation of the aerosolized medication, whereby the mask inlet valve closes and the exhalation outlet valve opens to allow the patient's exhalation to escape to the surrounding environment external to the apparatus.

FIG. 3 is a plan view of the preferred embodiment of the one-piece formable, flat-patterned sheet, including the thin film membrane valve seats attached thereto, from which the functional apparatus is formed.

FIGS. 4A-4C are views of various steps for forming a chamber forming portion of the preferred embodiment of the flat pattern in order to assemble the functional apparatus.

FIGS. 4D-4E are views of various steps for forming a mask forming portion of the preferred embodiment of the flat pattern in order to assemble the functional apparatus.

FIG. 4F is a drawing of the preferred embodiment of the apparatus in its fully assembled, functional form.

### DETAILED DESCRIPTION

While the present invention is capable of embodiment in various forms, there is shown in the drawings, and will be hereinafter described, one or more presently preferred embodiments with the understanding that the present disclosure is to be considered as an exemplification of the invention, and is not intended to limit the invention to the specific embodiments illustrated herein. Headings are provided for convenience only and are not to be construed to limit the invention in any way. Embodiments illustrated under any heading may be combined with embodiments illustrated under any other heading.

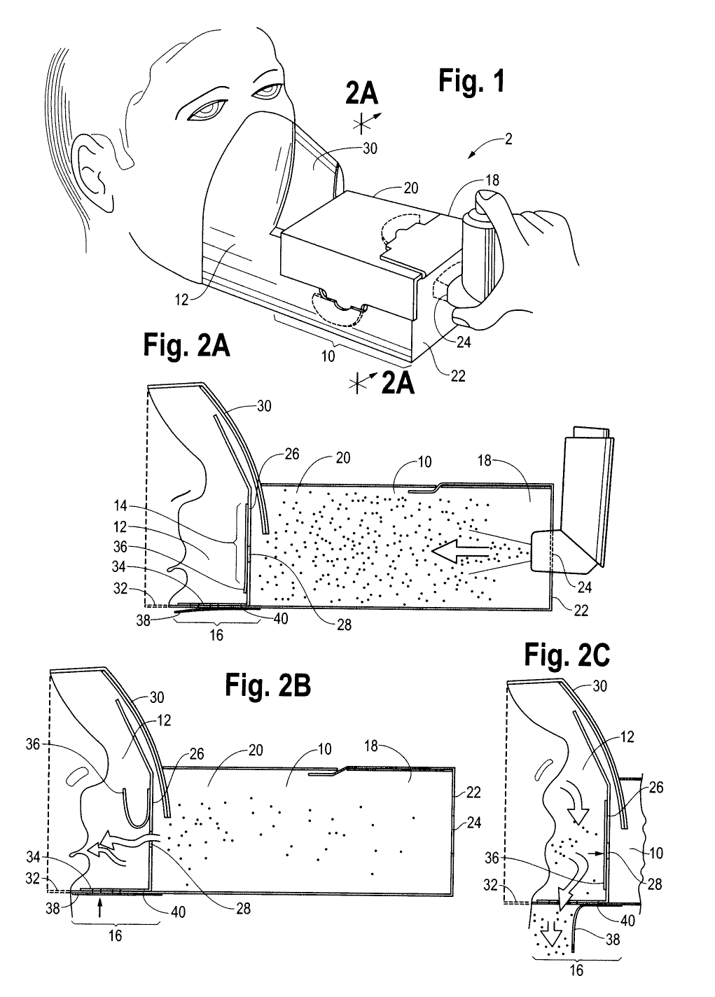

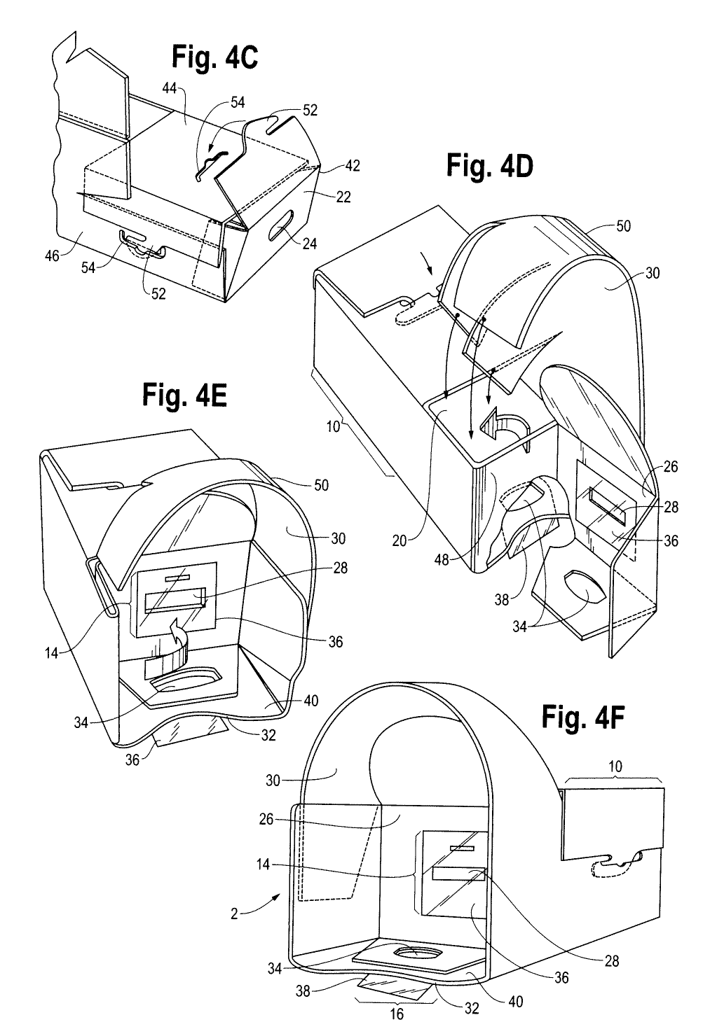

Referring to FIGS. 1 and 4F, a one-piece, foldable mask and holding chamber apparatus 2 for use with aerosolized medications is disclosed. The device is intended to improve the delivery of aerosolized particles of medication to the lungs of a patient such as an infant, young child, or any other individual who may have difficulties coordinating the simultaneous administering of aerosolized medications with the taking of a deep inhalation of such medications. In the preferred embodiment, the one-piece foldable mask and holding chamber apparatus 2 includes a single, flat-pattern sheet of material 4 that can be bent and folded to form a holding chamber 10, an integrated respiration mask 12, a one-way mask inlet valve 14, and a one-way exhalation outlet valve 16. In alternate embodiments, two separate flat-patterned sheets of material can be bent and folded to form a separate mask and separate holding chamber that may be connected to form the one-piece foldable mask and holding chamber apparatus 2.

Referring to FIGS. 1-2B, in the preferred embodiment, the holding chamber 10 is a substantially closed rectangular container having a first end 18 and a second end 20. The function of the holding chamber 10 is to accept aerosolized medication and hold it in aerosolized form until a patient inhales the medication into their lungs. The first end 18 of the holding chamber 10 contains a substantially closed first end-wall 22 containing an aerosolized medication insertion port 24 disposed therein. The medication insertion port 24 is an opening in the first end-wall 22 of the holding chamber 10 that is configured to receive a device for releasing aerosolized medications, such as the mouthpiece or nozzle of an MDI inhaler or a nebulizer.

The second end 20 of the holding chamber 10 contains a substantially closed common dividing wall 26 that contains a mask inlet valve aperture 28 disposed therein. In a preferred embodiment, the respiration mask 12 is integrally connected to the holding chamber 10 at the second end 20 of the holding chamber 10, with the common dividing wall 26 separating the mask 12 from the holding chamber 10.

In the preferred embodiment, the dimensions of the rectangular holding chamber 10 are 11 cm×5 cm×7 cm, which is sized for the inhalation volume of a small child's lungs. Alternatively, the dimensions of the holding chamber 10 can be enlarged to accommodate the larger inhalation volume of an adult. In yet additional alternate embodiments, the holding chamber 10 can take any alternate shape or have any alternate dimensions that will allow the holding chamber 10 to receive aerosolized medication from an MDI or other aerosolized medication producing device, and hold it until a patient inhales the medication into his lungs. Furthermore, while the embodiments depicted in the accompanying drawing figures show the first end 18 being located opposite the second end 20, this depiction should not be read to limit the location of the first end 18 and the medication insertion port 24 to only the location opposite the second end 20. Finally, the holding chamber 10 may also contain an optional transparent window to allow the aerosolized medication particles inside the holding chamber 10 to be viewed by an observer outside of the holding chamber 10.

In the preferred embodiment, the mask 12 is integrally connected to the second end 20 of the holding chamber 10, and includes a nasal hood 30, a contoured chin sealing cutout 32, the mask inlet valve aperture 28, and an exhalation outlet valve aperture 34 adjacent to the chin sealing cutout 32. A one-way mask inlet valve 14 is disposed in the common dividing wall 26 that separates the mask and holding chamber. The inlet valve 14 includes the mask inlet valve aperture 28 and a thin film, inlet valve membrane 36 attached at an upper edge of the inlet valve membrane 36 to the mask side of the dividing wall 26 immediately above the inlet valve aperture 28 disposed therein. The remainder of the inlet valve membrane 36 hangs free. The inlet valve membrane 36 is sized to have an area greater than that of the inlet valve aperture 28, and is positioned such that the remainder of the inlet valve membrane 36 that is hanging free will completely cover the inlet valve aperture 28. The inlet valve membrane 36 will thus flex at its free end and either open or close the mask inlet valve 14 depending on the changes in pressure inside the mask 12 due to the inhalation or exhalation of the patient.

A one-way mask exhalation outlet valve 16 is disposed in the mask's lower external sidewall wall 40 adjacent to the chin cutout 32. The exhalation outlet valve 16 similarly includes the outlet valve aperture 34 and a thin film, outlet valve membrane 38 attached at one edge thereof to an exterior surface of the mask's lower external sidewall 40, immediately adjacent to the exhalation outlet valve aperture 34, such that the remainder of the outlet valve membrane 38 will completely cover the aperture 34. Thus, the membrane 38 will similarly flex at its free end and either open or close the exhalation outlet valve 16, depending on the changes in pressure inside the mask 12 due to inhalation or exhalation of the patient.

Preferably, the membranes 36 and 38 are each attached to the mask surfaces by way of an adhesive disposed on one edge of each of the membranes 36 and 38, however in alternate embodiments the membranes 36 and 38 may be attached to the mask surfaces by staples, tabs and slots, or any other method that will secure the membranes in the proper location and still allow the membranes to flex in order to properly open or close the valves 14 and 16. In addition, the preferred embodiment includes a stamped cut located adjacent to the valve apertures 28 and 34 in order to indicate the proper attachment location of the valve membranes 36 and 38. Alternatively, the proper attachment location for the membranes 36 and 38 may be indicated by marking the locations on the mask surfaces with various text or symbols. The size and weight of the material that the membranes 36 and 38 are made from can be adjusted to accommodate different breathing forces of various patients. In alternate embodiments, the valves 14 and 16 can be any type of one-way valve that allows gases to flow there through in only one direction.

Referring to FIG. 1, in the preferred embodiment, the mask 12 is configured to cover the nose and mouth of a child patient. The mask 12 is also configured to form a substantial seal over the nose and mouth of the patient, which is sufficient to allow the patient to draw the aerosolized medication contained in the holding chamber 10 through the inlet valve 14, into the mask 12, and ultimately into the patient's lungs upon inhalation by the patient. While the preferred embodiment describes the mask 12 as being sized to cover and seal over the mouth and nose of a child, in alternate embodiments, the mask 12 can be sized to accommodate the face of any patient, from an infant to an adult. Additionally, in another embodiment, the edges of the mask 12 that contact the face of the patient can be coated with a flexible material such as a foam gasket, rubber tubing, or any other flexible material, in order to improve the ability of the mask 12 to form a substantial seal with the face of a patient.

Referring to FIGS. 1-2C, in operation, the fully formed one-piece foldable mask and holding chamber apparatus 2 is positioned such that the mask 12 is fully covering the patient's nose and mouth, and forms a substantial seal with the skin of the patient's face. Specifically, in the preferred embodiment, the nasal hood 30 of the mask 12 is covering the patient's nose and mouth and the patient's chin is pressed into the chin sealing cutout 32. If any air gaps occur between the edge of the mask 12 and the patient's face, the nasal hood 30 can be squeezed to adjust the edges of the mask 12 to match the contours of the patient's face, thus eliminating the air gaps that prevented a substantial seal from being formed between the mask 12 and the patient's face.

Next, referring to FIG. 2A, a mouth piece or nozzle of an MDI inhaler, or other similar device that produces aerosolized medications, is inserted into the medication insertion port 24 where the aerosolized medication producing device is then triggered, thus introducing the aerosolized medication into the holding chamber 10.

Referring to FIGS. 2B and 2C, the patient then breathes normally. When the patient inhales, the pressure inside the mask 12 drops below both the pressure inside the holding chamber 10 and the surrounding, external atmospheric pressure, thus forcing the exhalation outlet valve 16 to close and the mask inlet valve 14 to open. With the inlet valve 14 open and the patient inhaling, the aerosolized medication passes from the holding chamber 10, through the inlet valve 14 to the interior of the mask 12, and into the patient's mouth to reach his lungs (See FIG. 2B). When the patient exhales, the pressure inside the mask 12 rises above the pressure inside both the holding chamber 10 and the surrounding, external atmospheric pressure, thus forcing the mask inlet valve 14 to close and the exhalation outlet valve 16 to open, wherein the patient's exhalation is expelled to the surrounding atmosphere (See FIG. 2C). The process is then repeated as needed.

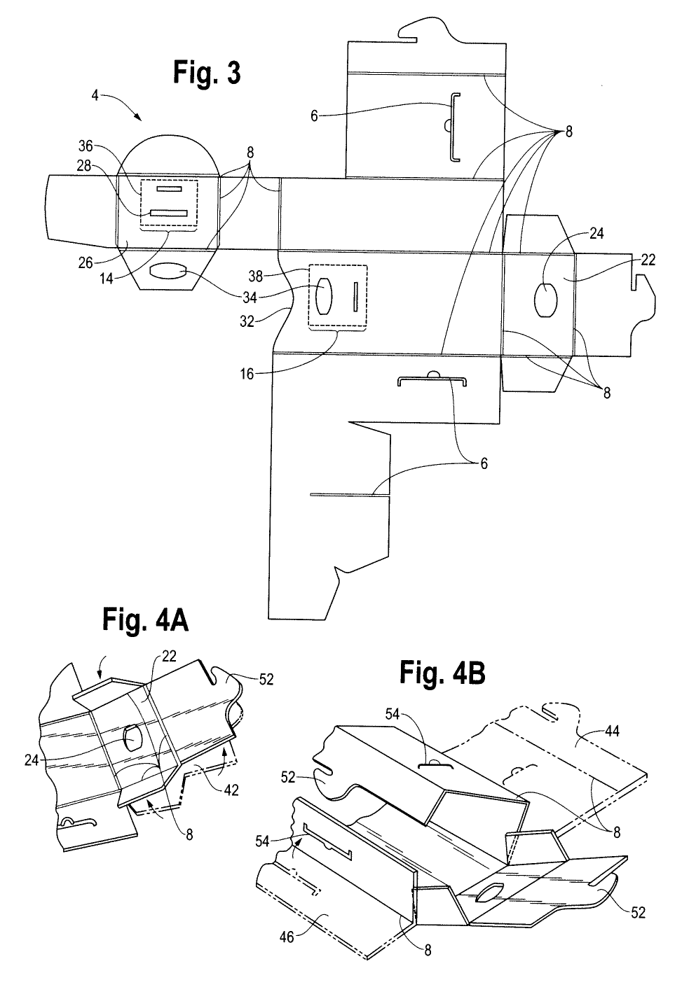

In order to use the one-piece, foldable mask and holding chamber apparatus 2 disclosed above, it must first be formed from a flat-patterned sheet 4 into its final, usable apparatus form. Referring to FIG. 3, the one-piece, foldable mask and holding chamber apparatus 2 begins as a single sheet of material cut into a flat pattern 4 having a predetermined shape. The flat-pattern 4 has a series of cuts 6 and creases 8 made in it at specific locations to aid in its assembly, as well as an opening 24 that will become the aerosolized medication insertion port 24 and several apertures 28 and 34 that will form the two valves 14 and 16. While the preferred embodiment of the flat pattern is disclosed and depicted in FIG. 3, it should be understood that variations to the size and shape of this flat-pattern 4 can be made without affecting the scope of the disclosure. This flat pattern 4 can either be shipped as a flat pattern 4 to the end user or the institution, hospital, or clinic that will ultimately put the apparatus 2 to use, or it can alternatively be shipped to a middleman who will form the flat pattern 4 into its final, usable apparatus 2 shape and then send it on to the end user. Alternatively, in addition to shipping the flat pattern 4 in its flat, unformed state or in its final, usable apparatus 2 state, the flat pattern 4 can be shipped to the end user or middleman in a partially folded state, if it is deemed appropriate or necessary to do so. If the flat pattern 4 is sent to the end user, it will then be assembled by a patient, a doctor, or any other individual, by folding the flat-pattern 4 into the usable shape of the one-piece, foldable mask and holding chamber apparatus 2, prior to use in connection with an MDI or other device that provides medicated aerosol for inhalation. The preferred embodiment of the final, usable shape of the one-piece, foldable mask and holding chamber apparatus 2 is shown in FIGS. 1 and 4F. In the preferred embodiment, the flat pattern 4 is made from a durable paperboard or cardboard material, but in alternate embodiments the flat pattern 4 may also be made from plastic, Tyvek, or any other suitable durable material that can be bent or formed and will hold its bent or formed shape.

Referring to FIGS. 4A-4C, in the preferred embodiment, a chamber forming portion of the flat pattern 4 is first formed by folding a first segment 42 of the flat pattern 4 along predefined creased fold lines 8 in order to form the first end-wall 22 of the holding chamber 10, which contains the medication insertion port 24 disposed therein. Next, a second segment 44 and third segment 46 of the flat pattern 4 are folded along additional predefined creased fold lines 8 in order to form the sidewalls of the holding chamber 10. The shape and integrity of the sidewalls is maintained by tucking an assembly retention tab 52 on the second segment 44 into a tab engagement slot 54 cut into the third segment 46. Additionally, the first end-wall 22 is secured in place by tucking an assembly retention tab 52 on the first segment 42 into a tab engagement slot 54 cut into the second segment. Alternatively, the number of tabs and slots can be increased or decreased as needed to maintain the formed shape of the holding chamber 10 without affecting the scope of this disclosure. Furthermore, the tab 52 on the second segment 44 and the mating slot 54 on the third segment 46 can be interchanged without affecting the scope of this disclosure.

Next, referring to FIGS. 4D-4F, in the preferred embodiment the mask forming portion of the flat pattern 4 is formed to create the respiration mask 12 by folding a fourth segment 48 of the flat pattern 4 to form the common dividing wall 26 containing the inlet valve aperture 28 and to close off the second end 20 of the holding chamber 10. A fifth segment 50 of the flat pattern 4 is next bended over to form the nasal hood 30 of the respiration mask 12. The fourth and fifth segments 48 and 50 are held in place by friction occurring between the folded segments and by tucking three flaps from the nasal hood 30 into slots formed by folding the various segments (See FIGS. 4D and 4E).

The mask inlet valve 14 is formed by attaching the upper edge of the thin film, inlet valve membrane 36 to the mask side of the dividing wall 26 immediately above the inlet valve aperture 28 disposed therein. The remainder of the membrane 36 is then allowed to hang free. The membrane 36 is sized to have an area greater than that of the aperture 28, and is positioned such that the remainder of the membrane 36 that is hanging free will completely cover the inlet valve aperture 28.

The exhalation outlet valve 16 is similarly formed by attaching a thin film, outlet valve membrane 38 at one edge thereof to the exterior surface of the of the mask's lower external wall 40, immediately adjacent to the exhalation valve outlet aperture 34, such that the remainder of the membrane 38 can completely cover the aperture 34.

In alternate embodiments, the steps taken to form the final usable apparatus 2 may be performed in any order, including, but not limited to, first forming the mask 12 and then forming the holding chamber 10, or first attaching the inlet and outlet membranes 36 and 38 to the flat pattern 4 in their proper locations, then forming either the holding chamber 10 or the respiration mask 12. Additionally, alternate methods of holding the various segments in place may be utilized such as taping the segments in place as opposed to using assembly retention tabs 52 and tab engagement slots 54.

One major benefit of a single, flat-pattern sheet of formable material 4 that can be formed to create a foldable mask and holding chamber apparatus 2 is that it costs significantly less to manufacture and significantly less to purchase than existing systems manufactured today, but yet it is still as effective as existing plastic or metal spacer and mask systems currently available on the market. This means that this apparatus is an affordable solution to aid those with acute respiratory diseases, such as asthma, that can be made available to individuals located in poorer, less economically developed countries and those individuals located in poor rural areas far from clinics or hospitals. The apparatus' 2, in either their assembled, usable form or in their flat-pattern form, can be kept and used at hospitals and clinics without having to worry about having to pay for expensive, high cost systems. Alternatively the apparatus' can be given to the individual patient to take home with them without a significant expense to the hospital or clinic. Furthermore, individuals can keep several of the flat patterned sheets, or several formed apparatus', in various locations like their home, office or job, their car, or in a travel bag to be used at any time without a large cost and without having to purchase multiple expensive systems in use today. Another major benefit of a single, flat-pattern sheet of formable material 4 that can be formed to create a foldable mask and holding chamber apparatus 2 is that it is compact as well as inexpensive. Because the apparatus begins as a flat pattern, the flat pattern and even the formed apparatus are compact and can be easily carried in a backpack or purse by the patient or end user.

As a person skilled in the art will recognize from the previous detailed description and from the figures and claims, modifications and changes can be made to the preferred embodiment of the invention without departing from the scope of the disclosure and claims.

What is claimed is:

1. An inhalation apparatus for use with aerosolized medications comprising:
a formable flat-patterned sheet, formed to bound a substantially closed aerosolized medication holding chamber having a first end and a second end, and a respiration mask integrally connected to said second end of said holding chamber;
an aerosolized medication insertion port disposed in said first end of said holding chamber and configured to receive a device for releasing aerosolized medications;
a one-way mask inlet valve disposed in a common dividing wall between said second end of said holding chamber and said respiration mask for providing a passage for said aerosolized medications from said chamber into said mask; and
a one-way mask exhalation outlet valve disposed in said mask for allowing exhalations from said patient's nose or mouth to escape to a surrounding environment external to said apparatus.

2. The inhalation apparatus of claim 1, wherein said apparatus further comprises at least one assembly retention tab on said flat-pattern sheet and at least one tab engagement slot, wherein said tab tucks into said corresponding tab engagement slot in said flat-pattern sheet for forming said medication holding chamber and said mask.

3. The inhalation apparatus of claim 2, wherein said inlet valve further comprises an inlet aperture, disposed in said common dividing wall between said chamber and said mask, and a thin valve membrane having an upper edge, wherein said membrane is attached at said upper edge to said common dividing wall on an inner surface of said mask above said aperture, and wherein the remainder of said membrane hangs free and is positioned to completely cover said inlet aperture.

4. The inhalation apparatus of claim 2, wherein said exhalation outlet valve further comprises an outlet aperture disposed in an external wall of said mask, and a thin valve membrane having a forward edge, wherein said membrane is attached at said forward edge to an external surface of said mask adjacent to said outlet aperture, and wherein the remainder of said membrane hangs free and is positioned to completely cover said outlet aperture.

5. The inhalation apparatus of claim 2, wherein said mask is configured to form a seal around a patient's nose and mouth,
wherein when said patient places said mask around his nose and mouth, introduces aerosolized medication into said holding chamber through said medication insertion port, and inhales, said inlet valve opens, said outlet valve closes, and said medication passes from said holding chamber through said inlet valve, into said mask, and into said patient's lungs, and
wherein when said patient exhales into an said mask, said inlet valve closes and said mask exhalation outlet valve opens allowing said patient's exhalation to escape to said surrounding environment.

6. An inhalation apparatus for use with aerosolized medications comprising:
an aerosolized medication holding chamber having a first end and a second end, wherein said first end includes an aerosolized medication insertion port configured to receive a device for releasing aerosolized medications;
a respiration mask integrally connected to said second end of said holding chamber, wherein said mask and integrally connected holding chamber are formable from a single flat-pattern sheet of durable material, and wherein said mask is configured to form seal around a patient's nose and mouth;
a one-way mask inlet valve disposed in a common dividing wall between said second end of said holding chamber and said respiration mask for providing a passage for said aerosolized medications from said chamber into, but not out of, said mask; and
a one-way mask exhalation outlet valve disposed in said mask for allowing exhalations from said patient's nose or mouth to escape to a surrounding environment external to said apparatus.

7. The inhalation apparatus of claim 6, wherein said apparatus further comprises at least one engagement tab on said flat-pattern sheet, wherein said tab tucks into at least one corresponding tab engagement slot in said flat-pattern sheet for forming said medication holding chamber and said mask.

8. The inhalation apparatus of claim 7, wherein said inlet valve further comprises an aperture disposed in said common dividing wall between said chamber and said mask, and a thin membrane having an upper edge, wherein said membrane is attached at said upper edge to said common dividing wall on an inner surface of said mask above said aperture, and wherein the remainder of said membrane hangs free and is positioned to completely cover said aperture.

9. The inhalation apparatus of claim 7, wherein said exhalation outlet valve further comprises an aperture disposed in an external wall of said mask, and a thin membrane having a forward edge, wherein said membrane is attached at said forward edge to an external surface of said mask in front of said aperture, and wherein the remainder of said membrane hangs free and is positioned to completely cover said aperture.

10. The inhalation apparatus of claim 7, wherein said mask is configured to form a seal around a patient's nose and mouth,
wherein when said patient places said mask around his nose and mouth, introduces aerosolized medication into said holding chamber through said medication insertion port, and inhales, said inlet valve opens, said outlet valve closes, and said medication passes from said holding chamber through said inlet valve, into said mask, and into said patient's lungs, and
wherein when said patient exhales into said mask, said inlet valve closes and said mask exhalation outlet valve opens allowing said patient's exhalation to escape to said surrounding environment.

11. An inhalation apparatus for use with aerosolized medications comprising:
a first formable flat-patterned sheet, formed to bound a substantially closed aerosolized medication holding chamber having a first end and a second end,
a second formable flat-patterned sheet, formed to bound a respiration mask, wherein said respiration mask is connected to said second end of said holding chamber;
an aerosolized medication insertion port disposed in said first end of said holding chamber and configured to receive a device for releasing aerosolized medications;
a one-way mask inlet valve disposed between said second end of said holding chamber and said respiration mask for providing a passage for said aerosolized medications from said chamber into said mask; and
a one-way mask exhalation outlet valve disposed in said mask for allowing exhalations from said patient's nose or mouth to escape to a surrounding environment external to said apparatus.

12. The inhalation apparatus of claim 11, wherein said apparatus further comprises at least one assembly retention tab on said first flat-pattern sheet and at least one tab engagement slot, wherein said tab tucks into said corresponding tab engagement slot in said flat-pattern sheet for forming said medication holding chamber.

13. The inhalation apparatus of claim 11, wherein said apparatus further comprises at least one assembly retention tab on said second flat-pattern sheet and at least one tab engagement slot, wherein said tab tucks into said corresponding tab engagement slot in said flat-pattern sheet for forming said respiration mask.

14. The inhalation apparatus of claim 11, wherein said inlet valve further comprises at least one inlet aperture, disposed between said chamber and said mask, and at least one thin valve membrane having an upper edge, wherein said membrane is attached at said upper edge on an inner surface of said mask above said aperture, and wherein the remainder of said membrane hangs free and is positioned to completely cover said at least one inlet aperture, and
wherein said exhalation outlet valve further comprises an outlet aperture disposed in an external wall of said mask, and a thin valve membrane having a forward edge, wherein said membrane is attached at said forward edge to an external surface of said mask adjacent to said outlet aperture, and wherein the remainder of said membrane hangs free and is positioned to completely cover said outlet aperture.

15. A method of assembling an inhalation apparatus for use with aerosolized medications, comprising:
creating a specific flat-pattern from a single sheet of formable material, wherein said flat pattern includes a chamber forming portion, a mask forming portion, at least one inlet aperture, and at least one outlet aperture;
forming said chamber forming portion of said flat pattern to create an aerosolized medication holding chamber; and
forming said mask forming portion of said flat pattern to create a respiration mask connected to said holding chamber to assemble said inhalation apparatus.

16. The method of assembling an inhalation apparatus of claim 11, wherein
forming said chamber forming portion further comprises:
folding a first segment of said flat pattern to form a first end of said holding chamber including an aerosolized medication insertion port; and
folding a second and third segment of said flat pattern to form a plurality of sidewalls of said holding chamber and said mask.

17. The method of assembling an inhalation apparatus of claim 12, wherein forming said chamber forming portion further comprises inserting at least one assembly engagement tab into a corresponding tab engagement slot so as to securely maintain the formed shape of said holding chamber.

18. The method of assembling an inhalation apparatus of claim 11, wherein forming said mask forming portion further comprises:
folding a fourth segment of said flat pattern so as to form a common dividing wall containing said inlet aperture disposed therein, said common dividing wall separating said holding chamber and said respiration mask, and to close off a second end of said holding chamber;
bending a fifth segment of said flat pattern to form a nasal hood of said mask.

19. The method of assembling an inhalation apparatus of claim 14, wherein said method further comprises attaching an inlet valve membrane to said common dividing wall on an inner surface of said mask such that said inlet aperture and inlet valve membrane are configured to act as a one way inlet valve to allow aerosolized medication to pass from said medication holding chamber into an internal volume of said mask;

20. The method of assembling an inhalation apparatus of claim 14, wherein said method further comprises attaching an outlet valve membrane to an external surface of said mask's external side wall such that said outlet aperture and outlet valve membrane are configured to act as a one way outlet valve to allow a patient's exhalations to escape from said internal space of said mask during use of said apparatus, and wherein an external sidewall of said mask includes an outlet aperture connecting an internal volume of said mask to an external surrounding environment and through which gas may escape.
